(19) United States
(12) Patent Application Publication
(10) Pub. No.: US 2014/0261438 A1
(43) Pub. Date: Sep. 18, 2014

(54) MULTI-POSITIONAL CONNECTOR FOR RESPIRATORY SUPPORT SYSTEMS

(71) Applicant: Darlene R. Mieskoski, Strongsville, OH (US)

(72) Inventor: Darlene R. Mieskoski, Strongsville, OH (US)

(21) Appl. No.: 13/843,017

(22) Filed: Mar. 15, 2013

(51) Int. Cl. A61M 16/08

(52) U.S. Cl. CPC A61M 16/0816
USPC 128/206.21

(57) ABSTRACT

A multi-positional connector for respiratory support systems that enables the attachment and detachment of an accessory device to a respiratory support system in a manner that does not require an interruption in a patient's oxygen supply but does allow the patient to easily. change positions when the accessory device is a nebulizer. The device has four components that are push-fit together and are able to rotate relative to each other.

### BACKGROUND OF THE INVENTION

1. Field of the Invention

The present invention relates to an oxygen flow related delivery system used to supplement a patient's respiration and, more specifically, to a multi-port connector used to connect an accessory device to a patient's oxygen mask thereby allowing attachment and detachment of other oxygen devices without interruption of continuous respiratory support.

2. Description of Related Art

Respiratory support systems, which assist a patient in maintaining adequate blood oxygenation levels without overtaxing the patient's heart and lungs, are well known in the art. Typically, such systems consist of a source of oxygen that is supplied via a tube to a respiratory mask secured over a portion of a patient's face. Often critically ill patients require continuous respiratory support from such systems and are severely stressed when that support is interrupted.

However, while a patient is attached to a respiratory support system, it is commonly necessary to periodically provide medications directly into the patient's lungs via a nebulizer. A nebulizer delivers liquid medication via a fine mist which enables the medication to be absorbed through the lung's thin membranes. For patients on respiratory support systems, nebulized medication is typically delivered to the lungs by disconnecting the respiratory system and having the patient breathe through a mouthpiece or mask attached to the nebulizer.

Examples of nebulizers with dedicated mouthpieces or masks are provided by U.S. Pat. Nos. 5,277,175; 5,570,682; 5,752,502; 5,479,920; and 5,099,833, and U.S. Pat. App. Pub. No. 2002/0020412. The serious shortcoming associated with the devices taught by these patents is that each requires an interruption in a patient's oxygen supply for the period that his or her respiratory support mask is disconnected while using the nebulizer mouthpiece or mask. Consequently, the patient experiences a decrease in oxygen consumption while receiving the aerosolized medications, which commonly requires a period of ten to twenty minutes. The interruption in oxygen supply for this amount of time often results in hypoxemia and can require twenty minutes or more for the patient's blood oxygen levels to recover once the patient is reconnected to the respiratory support system. Significantly, however, such recovery periods are very stressful to patients, particularly elderly patients.

Patent applications have been filed for devices that avoid an interruption in oxygen supply by allowing both a pressurized source of oxygen and a nebulizer to be connected to a single mask or mouthpiece. Examples of such are provided by U.S. Pat. App. Pub. Nos. 2001/0035181; 2002/0002975; and 2002/0020409. However, while these devices may eliminate the interruption in oxygen supply currently required by use of a nebulizer, each has at least one drawback.

U.S. Pat. App. Pub. No. 2001/0035181 to Elkins discloses a rebreather nebulizer which shows a nebulizer and a separate source of pressurized oxygen connected to a collapsible bag which is in turn connected to a respiratory mask. The device allows a patient to simultaneously receive pressurized oxygen and medications via a nebulizer. However, when the nebulizer is not being used, the device does not allow it and the collapsible bag to be disconnected from the oxygen supply. Nor does the device allow for a patient to easily change positions when receiving medication via the nebulizer. A nebulizer must be in an upright position to function properly. But with this device, the nebulizer is connected to the base of the collapsible bag which is attached directly to a respiratory mask; therefore, the patient must sit upright when using it.

Similarly U.S. Pat. App. Pub. Nos. 2002/0002975 to Power and 2002/0020409 to Kidwell et al., each disclose a device that allows for coupling of a nebulizer with a source of pressurized oxygen and a respiratory mask. However, neither of these devices allows a patient to easily change positions when receiving medication via a nebulizer. The patent application to Power discloses a three port connector in which a nebulizer is attached directly to a port on the underside of a conduit and, therefore, is structured such that a patient must be sitting up to use it. Likewise, the patent application to Kidwell et al., shows several embodiments of a three port connector in which a nebulizer is attached directly to a port on either the side or the underside of a conduit thereby preventing the nebulizer from being repositioned relative to the conduit and preventing a patient from changing positions.

Consequently, a need exists for a multi-port connector that allows for attachment and detachment of a nebulizer to a respiratory support system and for a patient to easily change positions when using such a connector. None of the above inventions and patents, taken either singly or in combination, is seen to describe the instant invention as claimed.

### SUMMARY OF THE INVENTION

The present invention is a multi-positional connector that allows for the attachment and detachment of an accessory device to a respiratory support system in a manner that does not require an interruption in the patient's oxygen supply but does allow the patient to easily change positions when the accessory device is a nebulizer. The device comprises standard sized parts that are push-fit together and are able to rotate relative to each other. It enables compatibility between multiple devices, which has not previously existed, thereby allowing for greater flow of either oxygen, air, or medication and also allowing for the use of vibration and resistance devices in the promotion of improved respiratory care. Notably, the multi-positional connector allows for the attachment of an accessory device supplied from a regulated gas source through an independent delivery system.

Accordingly, it is a principal object of the invention to provide a means for attaching and detaching an accessory device such as a nebulizer, flutter, EZ Pep, Thera Pep, metered dose inhaler or second source of gas to a standard respiratory mask. It is another object of the invention to allow a patient on a respiratory support system to avoid the risk of hypoxemia by providing the benefits of an accessory device, such as a nebulizer, without having to endure an interruption in oxygen supply.

It is a further object of the invention to provide a means of connecting a nebulizer to a respiratory mask wherein the effectiveness of the nebulizer is not compromised by patient's body position.

Still another object of the invention is to provide a connector that is universally adaptable to most respiratory support systems and masks.

Additionally, it is an object of the invention to provide improved elements and arrangements thereof in an apparatus for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental view of a multi-positional connector for a respiratory support system according to the present invention, as used by a patient in an upright position.

FIG. 2 is an environmental view of the multi-positional connector for a respiratory support system as used by a reclining patient.

FIG. 3 is an enlarged-scale, elevational view of the multi-positional connector for a respiratory support system connected to a respiratory mask.

FIG. 4 is an enlarged-scale elevational view of a multi-positional connector for a respiratory support system.

FIG. 5 is an exploded view of a multi-positional connector for a respiratory support system shown in FIG. 4.

FIG. 6 is an elevational view of an alternative embodiment of a multi-positional connector.

FIG. 7 is an exploded view of the multi-positional connector shown in FIG. 6.

FIG. 8 is an elevation view of the flex tube component of the multi-positional connector shown in FIG. 6.

Similar reference characters denote corresponding features consistently throughout the attached drawings.

### DETAILED DESCRIPTION OF THE PREFERRED EMBODIMENTS

The present invention is a multi-positional connector that enables the attachment and detachment of an accessory device to a respiratory mask without requiring an interruption in a patient's supply of oxygen and, when the accessory device is a nebulizer, allows the nebulizer to function properly regardless of the patient's body position.

Referring to the drawings, FIGS. 1 through 5 illustrate the preferred embodiment of the present invention, and FIGS. 6 through 8 illustrate an alternative preferred embodiment.

FIGS. 1 and 2 show environmental views of the preferred embodiment 20 as used by a patient wearing a respiratory mask. In FIG. 1, the patient is sitting in an upright position and in FIG. 2, the patient is reclining. It will be appreciated from the views that a nebulizer 12, attached to the multi-positional connector 20, remains substantially upright regardless of the patient's body position.

FIG. 3 shows an elevated view of the multi-positional connector 20 connected to a respiratory mask 10, a respiratory support system tube 11 and a nebulizer 12.

FIG. 4 shows the four components of the multi-positional connector 20 assembled together. A tubular T-shaped connector 21, having two ends and a lateral port 25, is attached to three other components. The T-shaped connector 21 has an opening at each of its ends and at the end of its lateral port 25. The body of the T-shaped connector 21 defines a passageway between the openings, allowing each opening to communicate with the other two.

A mask adapter 22 is attached to one end of the T-shaped connector 21 via push fit engagement. The mask adapter 22 is tubular and can be rotated relative to the T-shaped connector 21 by twisting with one's fingers.

A respiratory tubing adapter 23 is attached, via push-fit engagement, to the other end of the T-shaped connector 21. The respiratory tubing adapter is tubular with a one-step change in diameter. While its upper portion 26 is dimensioned to fit securely around the end of the T-shaped connector, its lower portion 27 is dimensioned to fit within standard sized respiratory support system tubing. The respiratory tubing adapter can also be rotated relative to the T-shaped connector 21 by twisting with one's fingers. A lateral port tube 24 is attached to the end of the T-shaped connector's lateral port 25 via push fit engagement. It 24 is tubular with two ends, a 90° bend and a one-step change in diameter near its distal end 29. The distal end 29 is dimensioned to fit within a standard sized nebulizer port. The lateral port tube can also be rotated relative to the T-shaped connector 21 by twisting with one's fingers. When not

connecting a nebulizer or other accessory device, the distal end 29 is covered with a cap (not shown) dimensioned to fit securely over it.

FIG. 5 shows an exploded view of FIG. 4. The four components of the multi-positional connector 20 include the mask adapter 22, the T-shaped connector 21, the respiratory tubing adapter 23 and the lateral port tube 24. Both the respiratory tubing adapter 23 and the lateral port tube 24 include a one-step change in diameter such that the lower portion 27 of the respiratory tubing adapter 23 fits securely in standard sized respiratory tubing and the end 29 of the lateral port tube 24 fits securely in a standard sized nebulizer port.

FIGS. 6, 7 and 8 show an alternative embodiment 40 of the multi-positional connector according to the present invention which differs from the preferred embodiment in only one respect. In the alternative embodiment the lateral port tube 44 is flexible and can be bent 90° in any direction. A section of the lateral port tube 44 includes eight pleats 51 such that diameter of each pleat first tapers outward for a certain distance and then tapers inward for the same distance, each pleat 51 immediately followed by another. The pleats 51 are uniformed in outer most and innermost diameter and in length and degree of taper. When the lateral port tube 44 is bent, as shown in FIG. 8, the pleats gather on the interior aspect of the bend and stretch on the exterior aspect of the bend, thereby allowing the tube to bend without diminution of its passageway.

Like the preferred embodiment, the alternative embodiment includes a T-shaped connector 41, a mask adapter 42 and a respiratory tubing adapter 43, all of which function in a manner identical with that of the preferred embodiment.

In both the preferred and alternative embodiments, a one-way valve (not shown) may be positioned inside the lateral port thereby providing an automatic seal of the lateral port when an accessory device is removed.

It is to be understood that the present invention is not limited to the embodiments described above, but encompasses any and all embodiments within the scope of the following claims.

I claim:

1. A multi-positional connector comprising:
a tubular housing having a first end, a second end, and a lateral port;
said tubular housing defining a first opening at said first end, a second opening at said second end, a passageway between said first and second openings, and a passageway into said lateral port;
said lateral port being tubular with a first end, a second end, and a passageway between said first and said second ends;
and
said first end of said lateral port being proximal to said tubular housing, and said second end of said lateral port defining a third opening.

2. The multi-positional connector according to claim 1, wherein:
said first opening is shaped and dimensioned to attach to a standard size port opening on a respiratory mask by means of a push-fit engagement;
and
said second opening is shaped and dimensioned to attach to standard size respiratory support system tubing by means of push fit engagement;
whereby, when said first opening is attached to respiratory mask port, said tubular housing can be rotated relative to said respiratory mask port by applying torque to said tubular housing, and when said second opening is attached to respiratory support system tubing, said tubular housing can be rotated relative to said respiratory support system tubing by applying torque to said tubular housing.

3. The multi-positional connector according to claim 2, wherein:
said first opening has an inside diameter of 22 millimeters;
and
said second opening has an outside diameter of 15 millimeters.
